(19) United States
(12) Patent Application Publication
(10) Pub. No.: US 2015/0293086 A1
(43) Pub. Date: Oct. 15, 2015

(54) LATERAL FLOW IMMUNOASSAY

(71) Applicant: ABREOS BIOSCIENCES, INC., San Diego, CA (US)

(72) Inventors: Bradley Todd MESSMER, San Diego, CA (US); Peter HABERZ, San Diego, CA (US)

(21) Appl. No.: 14/686,578

(22) Filed: Apr. 14, 2015

Related U.S. Application Data
(60) Provisional application No. 61/979,123, filed on Apr. 14, 2014.

(51) Int. Cl. G01N 33/543; G01N 33/68; G01N 33/94; G01N 33/558

(52) U.S. Cl. CPC G01N 33/54386; G01N 33/558; G01N 33/6854; G01N 33/94

(57) ABSTRACT

Disclosed is a test device for determining the presence or integrity of a biologic comprising a sample pad for receiving the biologic, a conjugate pad, and a test membrane comprising at least one test line comprising an immobilized mimetope, and methods of using the same.

### RELATED APPLICATIONS

The present application claims the benefit of U.S. Provisional Application Ser. No. 61/979,123, filed on Apr. 14, 2014, by Bradley T. MESSMER et al. and entitled “LATERAL FLOW IMMUNOASSAY,” the entire disclosure of which is incorporated herein by reference, including the drawings.

### GOVERNMENT RIGHTS

The subject matter disclosed herein was made with government support under grants 1R41CA192697-01 and 1R43CA183241-01 awarded by the National Institutes of Health Small Business Innovation Research (NIH-SBIR). The government has certain rights in the disclosed subject matter.

### FIELD OF THE INVENTION

The present invention is in the field of test devices, and more specifically in the field of lateral flow test devices.

### BACKGROUND OF THE DISCLOSURE

Biologics are valuable therapeutics for treating cancers and other diseases. As they are among the most expensive pharmaceuticals produced and increasingly prescribed, there have been a growing number of incidents involving counterfeit biologics, such as counterfeit antibodies. These economics create a considerable incentive for counterfeit or other deceptions. The problem of counterfeit medications is growing rapidly. The Center for Medicines in the Public Interest estimates that the worldwide trade for counterfeit medicines exceeds $75 billion. This number is $25 billion more than the illicit drug trade. Therefore, there is a concern that organized crime will become more active in this area.

Furthermore, biologics are sensitive to environmental conditions and improper storage or handling at any point in the supply chain. Improper handling of a biologic, such as an antibody, could reduce or inactivate their ability to bind the target antigen and mitigate their therapeutic effect. At present, there are no routine safeguards in place at the time of administration to ensure that a patient is receiving the correct, active therapy.

Thus, a need remains to develop simple, rapid assays that verifies both the presence and integrity of appropriate antibody in the sample and that are amenable for use at any point in the supply chain, including bedside. Such an assay will provide patients and their physicians an extra degree of confidence that the correct, active therapeutic is administered.

### SUMMARY OF THE INVENTION

Disclosed herein is a test device for determining the presence or integrity of a biologic comprising a sample pad for receiving the biologic, a conjugate pad, and a test membrane comprising at least one test line comprising an immobilized mimetope.

Disclosed herein is a method of determining the integrity of a biologic comprising (a) contacting the biologic with a test device, wherein the test device comprises a sample pad for receiving the biologic, a conjugate pad, a test membrane comprising at least one test line comprising an immobilized mimetope, (b) determining whether at least one test line undergoes a color change, (c) determining, based on the color change, the activity of the biologic.

Disclosed herein is a kit comprising (a) a test device for determining the integrity of a biologic, the test device comprising a sample receiving pad, a conjugate pad, and a test membrane, the test membrane further comprising at least one test line and at least one control line, and (b) instructions for use thereof.

### BRIEF DESCRIPTION OF THE DRAWINGS

FIG. 1 illustrates one embodiment of the lateral flow assay disclosed herein. The top panel illustrates a schematic of the prototype. A drop of the biologic sample is placed on a sample pad and is wicked through the device. After binding the secondary detection antibody, conjugated to gold, the sample encounters the peptide, anti-light chain, and anti-secondary capture agents. The bottom panel illustrates possible outcome matrix for an antibody that is known to possess a kappa light chain.

FIG. 2 (A) illustrates one embodiment of the lateral flow assay validating that rituximab binding mimetope peptide function in immunoassays and competes with the native antigen (CD20). FIG. 2 (A) is a standard curve for rituximab by peptide based ELISA. Biotinylated peptides are bound onto neutravidin coated ELISA plates. Rituximab is diluted in TBST. Each value shows the mean (±S.D.) of triplicates. The solid line indicates the mean of the buffer control and the dashed line represents the mean +10 times the SD of the buffer control.

FIG. 2 (B) illustrates peptide inhibition of Chronic Lymphocytic Leukemia (CLL) cell staining. Fluorescently labeled rituximab is incubated with primary CLL cells and evaluated by flow cytometry (solid line). Weak staining for CD20 was observed because CLL cells have low levels of CD20. When peptide RTX-10 was added at a large molar excess (dashed lines), the cell labeling was largely abrogated. Control peptides had no effect (not shown). The shaded histogram represents CLL cells incubated with fluorescently labeled normal human IgG.

FIG. 3 illustrates one embodiment of the lateral flow immunoassay design and development path. The flow chart shows the design process for a lateral flow immunoassay.

FIG. 4 illustrates one embodiment of mimetope LFA for bevacizumab, trastuzumab, and rituximab. The sample comprising an antibody flows through a conjugate pad loaded with goat anti-human IgG attached to colloidal gold, then onto a membrane striped with the appropriate mimetope peptide, and finally an assay control. A false positive on one anti-lambda test line is shown by an asterisk.

FIG. 5 illustrates that the lateral flow assay disclosed herein has wide dynamic range. Samples tested were Rituximab at 0.0 mg/ml (negative control), 0.01 mg/ml, 0.1 mg/ml, 1.0 mg/ml, and 10 mg/ml (vial concentration). With increasing Rituximab concentration, the test line gets more intense.

FIG. 6 further illustrates that the lateral flow assay disclosed herein has a wide dynamic range. FIG. 6 shows one embodiment of the sandwich lateral flow assay with two test lines. The test lines comprise peptide mimetope striped at two different concentrations on the membrane. Samples tested are Rituximab at 0.0 mg/ml (negative control), 0.01 mg/ml, 0.1 mg/ml, 1.0 mg/ml, and 10 mg/ml (vial concentration). With increasing Rituximab concentration, the lower test line gets less intense whereas the upper test line gets more intense.

### DETAILED DESCRIPTION OF THE EMBODIMENTS

The terminology used herein is for the purpose of describing particular embodiments only and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, of a given value. Where particular values are described in the application and claims, unless otherwise stated the term “about” meaning within an acceptable error range for the particular value should be assumed.

Unless otherwise defined, all technical and scientific terms used herein have the same meaning as commonly understood by one of ordinary skill in the art to which this invention belongs.

Unless otherwise stated, the following terms used in this application, including the specification and claims, have definitions given below.

The terms “protein” as used herein refers to at least one sequence of amino acids linked by sequential peptide bonds, and is generally synonymous with “polypeptide.” In one embodiment, the amino acid sequence is one that occurs in nature. In other embodiments, the amino acid sequence is engineered by man. The term protein includes, but is not limited to, proteins having pharmaceutical, diagnostic, agricultural, and/or any of a variety of other properties that are useful in commercial, experimental, and/or other applications. In some embodiments, the protein is a protein therapeutic. The term “protein therapeutic” (or “therapeutic protein”) as used herein contemplates a protein that has a biological effect in the body, or on a region in the body on which it directly acts, or on a region of the body on which it remotely acts via intermediates, etc. Examples of therapeutic proteins are, but is not limited to, pharmaceutically or commercially relevant enzymes, receptors, receptor fusions, soluble receptors, soluble receptor fusions, antibodies (e.g., monoclonal and/or polyclonal antibodies), antigen-binding fragments of an antibody, Fc fusion proteins, SMIPs5 cytokines, hormones, regulatory factors, growth factors, coagulation/clotting factors, and antigen-binding agents. The above list of proteins is merely exemplary in nature, and is not intended to be a limiting recitation.

The term “antibody” as used herein contemplates a polypeptide or a protein complex that specifically binds an epitope of an antigen or mimetope thereof. An antibody includes an intact antibody, or a binding fragment thereof that competes with the intact antibody for specific binding and includes chimeric, humanized, fully human, and bispecific antibodies. Binding fragments include, but are not limited to, Fab, Fab′, F(ab′)2, Fv, and single-chain antibodies. In some embodiments, an antibody is referred to as an immunoglobulin and include the various classes and isotypes, such as IgA (IgA1 and IgA2), IgD, IgE, IgM, and IgG (IgG1, IgG3 and IgG4) etc. In some embodiments, the antibody is polyclonal or monoclonal. In some embodiments, the antibody is from any origin, such as mouse or human, including a chimeric antibody thereof. In some embodiments, the antibody is humanized.

Examples of antibodies include, but are not limited to, bevacizumab, trastuzumab, rituximab, abciximab, adalimumab, alemtuzumab, basiliximab, belimumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, raxibacumab, tocilizumab, tositumomab and ustekinumab. Other examples of antibodies include, but are not limited to, 3F8, abagovomab, abatacept, acz885, adecatumumab, afelimomab, aflibercept, afutuzumab, alacizumab, altumomab, anatumomab, anrukinzumab, apolizumab, arcitumomab, aselizumab, atlizumab, atorolimumab, bapineuzumab, bavituximab, bectumomab, belatacept, bertilimumab, besilesomab, biciromab, bivatuzumab, blinatumomab, cantuzumab, capromab, catumaxomab, cedelizumab, citatuzumab, cixutumumab, clenoliximab, cnto1275(=ustekinumab), cnto148(=golimumab), conatumumab, dacetuzumab, detumomab, dorlimomab, dorlixizumab, ecromeximab, edobacomab, edrecolomab, efungumab, elsilimomab, enlimomab, epitumomab, epratuzumab, erlizumab, ertumaxomab, etanercept, etaracizumab, exbivirumab, fanolesomab, faralimomab, felvizumab, figitumumab, fontolizumab, foravirumab, galiximab, gantenerumab, gavilimomab, gomiliximab, ibalizumab, igovomab, imciromab, inolimomab, inotuzumab ozogamicin, iratumumab, keliximab, labetuzumab, lebrilizumab, lemalesomab, lerdelimumab, lexatumumab, libivirurnab, lintuzumab, lucatumumab, lumiliximab, mapatumumab, maslimomab, matuzumab, mepolizumab, metelimumab, milatuzumab, minretumomab, mitumomab, morolimumab, motavizumab, myo-029, nacolomab, naptumomab, nebacumab, necitumumab, nerelimomab, nimotuzumab, nofetumomab, ocrelizumab, odulimomab, oportuzumab, oregovomab, otelixizumab, pagibaximab, panobacumab, pascolizumab, pemtumomab, pertuzumab, pexelizumab, pintumomab, priliximab, pritumumab, pro-140, rafivirumab, ramucirumab, regavirumab, reslizumab, rilonacept, robatumumab, rovelizumab, rozrolimupab, ruplizumab, satumomab, sevirumab, sibrotuzumab, siltuximab, siplizumab, solanezumab, sonepcizumab, sontuzumab, stamulurnab, sulesomab, tacatuzumab, tadocizumab, talizumab, tanezumab, tapliturnomab, tefibazumab, telimomab, tenatumomab, teneliximab, teplizumab, tgn1412, ticilimumab (=tremelimumab), tigatuzumab, tnx-355 (=ibalizumab), tnx-650, tnx-901 (=talizumab), toralizumab, tremelimumab, tucotuzumab, tuvirumab, urtoxazumab, vapaliximab, vedolizumab, veltuzumab, vepalimomab, visilizumab, volociximab, votumumab, zalutumumab, zanolimumab, ziralimumab, and zolimomab.

The term “antibody” as used herein also contemplates the biosimilar or second generation version of the monoclonal antibodies described herein.

The term “biological fluid” as used herein contemplates a liquid with biomolecules, bioparticles, blood, sweat, saliva, amniotic fluid, lacrimal fluid, or urine. Examples of biomolecules are, but not limited to, nucleic acids, peptides, and enzymes. Examples of bioparticles are, but not limited to, cells, organelles etc.

The term “mimetope” as used herein contemplates a compound that is recognized by the same binding molecule, such as an antibody, as a particular epitope but which has a different composition from the epitope. In one embodiment, the binding molecule is an antibody which recognizes (i.e., binds to) an epitope comprising a linear sequence of amino acids. A mimetope of this epitope comprises a different linear sequence of amino acids but which is still recognized by the same antibody. In one embodiment, mimetope includes a peptide epitope that is able to mimic the ability of an epitome to bind to an antibody.

The terms “polypeptide” and “peptide” are used broadly to refer to macromolecules comprising linear polymers of natural or synthetic amino acids. In some embodiments, polypeptides are derived naturally or synthetically by standard methods known in the art. While the terms polypeptide and peptide are synonymous, the term polypeptide, as used herein, generally refers to molecules of greater than 40 amino acids, while the term peptide generally refers to molecules of 2 to 40 amino acids.

The term “sample pad” as used herein generally refers to a hydrophilic element, such as a membrane that receives the antibody. In some embodiments, the sample pad is part of the conjugate pad, or a discrete pad positioned downstream of and in fluid communication with the conjugate pad. In some embodiments, the sample pad is useful for promoting the even and controlled distribution of the antibody onto the conjugate pad, controlling the rate at which the antibody enters the conjugate pad, or preventing flooding of the test device. In some embodiments, the sample pad material comprise of, but not limited to, for example, woven mesh or cellulose filters, glass fiber, mixed glass fiber and cellulose, man-made fiber, mixed fiber, surface modified plastic (polyester, polypropylene, or polyethylene), nitrocellulose, or graded density polyethersulfone (PES). In some embodiments, the sample pad is of the same or different material as the conjugate pad. In some embodiments, the sample pad is impregnated with agents to influence the flow rate of the sample, including, for example, protein detergents or surfactants, viscosity enhancers, signal enhancers, or buffer salts. In some embodiments, agents are added to the sample pad to prevent the detectable marker and antibody from binding nonspecifically to any downstream materials or to modify the chemical nature of the antibody so that it is compatible to complex at the test line. In some embodiments, the sample pad further comprises a buffer, pH calibrator, peptide, or antibody.

The term “conjugate pad” as contemplated herein, generally refers to a hydrophilic element, such as a membrane, containing a conjugate reagent such as colloidal gold, latex particles, enzymes, colored dyes, or paramagnetic or fluorescent particles. In some embodiments, the conjugate pad acts to ensure uniform transfer of the detectable marker and the antibody onto the test membrane. In some embodiments, the conjugate pad comprises, but not limited to, woven mesh or cellulose filters, glass fiber, mixed glass fiber and cellulose, man-made fiber, mixed fiber, surface modified plastic (polyester, polypropylene, or polyethylene), nitrocellulose, or graded density PES. In some embodiments, the conjugate pad is part of the sample pad, or a discrete pad positioned upstream of, and in fluid communication with the sample pad. In some embodiments, the conjugate pad is of the same or different material as the sample pad. In some embodiments, the conjugate pad further comprises an antibody. In one of these embodiments, the antibody is an immunoglobulin antibody.

The term “test line” as contemplated herein, refers to a band or zone on the test membrane that contains at least one mimetope peptide. The mimetope peptide is usually immobilized in a band or zone such that after reaction with the antibody-detectable marker complex, the band or zone produces an observable or measurable signal reflecting the presence or amount of antibody present in the sample. For exemplary purposes only, if the test device is intended to detect the presence or the amount of bevacizumab in a sample, then at least one test line will contain a mimetope peptide sequence specific for bevacizumab immobilized on the test membrane. In some embodiments, the test membrane contains one, two, three, four, or more test lines. In some embodiments, the test lines contain the same or different immobilized mimetopes. In some embodiments, more than one test line is applied for multi-analyte testing or for semi-quantitative evaluation. In some embodiments, the peptides in the test lines are conjugated to a protein. In an exemplary embodiment, the protein is a globular protein, such as but not limited to, fibronectin, albumin, recombinant capsid protein VP1 of the foot-and-mouth-disease virus (rVP1), recombinant capsid protein VP2 of the foot-and-mouth-disease virus (rVP2), recombinant capsid protein VP3 of the foot-and-mouth-disease virus (rVP3), or precursor protein P1 of VP1, VP2, VP3, and VP4. In other embodiments, the protein is a chimeric protein, a naturally occurring protein or a non-naturally occurring protein.

The term “control line” as used herein contemplates a band or zone on the test membrane used for, but not limited to, confirming negative test results, for comparison with at least one test line, determining incorrect antibody, inactive antibody, wrong antibody, non-antibody protein, or concluding failed experiments. In some embodiments, the control line comprises an antibody. In one of these embodiments, the antibody is an immunoglobulin antibody. The immunoglobulin antibody include various classes and isotypes, such as IgA (IgA1 and IgA2), IgD, IgE, IgM, and IgG (IgG1, IgG3 and IgG4) etc. The antibody may be a human antibody or a non-human antibody. The antibody may be naturally occurring or non-naturally occurring.

The term “wicking pad” as used herein contemplates an absorbent pad attached at the distal side of the test device. The wicking pad aids to maintain the flow of liquid to the end of the strip. In some embodiments, the wicking pad comprises, but not limited to, an absorbent material, cellulose filter, or any other material which acts to maintain the flow of liquid to the end of the strip.

It is a general object of the present disclosure to provide methods, devices and kits which is used to visually determine the presence or absence of a specific, biologic and/or to visually quantify or semi-quantify an amount of a specific, biologic. It is also a general object of the present disclosure to provide methods, devices and kits which is used to visually determine the presence or absence of a specific biologic and/or to visually quantify or semi-quantify an amount of a specific biologic. In some embodiments, it will be readily appreciated that other detection systems, optical or otherwise, is used in such tests.

In some embodiments, the term “biologic” as used herein refers to a virus, therapeutic serum, toxin, antitoxin, vaccine, blood, blood component or derivative, allergenic product, protein, or analogous product, or arsphenamine or derivative of arsphenamine (or any other trivalent organic arsenic compound), applicable to the prevention, treatment, or cure of a disease or condition of human beings. In other embodiments, the term biologic has the definition of biological product set forth by the Food and Drug Administration (http://www.fda.gov/Drugs/DevelopmentApprovalProces s/%20HowDrugsareDevelopedandAppr oved/ApprovalApplications/TherapeuticBiologicApplications/ucm113522.htm).

In an exemplary aspect, the biologic is a protein. In those aspects, the present disclosure relates to detection of proteins. Proteins are typically used in therapeutic applications, such as in the treatment of cancer. In some embodiments, as used herein, the protein is from any origin, such as mouse, rabbit, pig, horse, dog, or human, including a chimeric protein thereof. In some embodiments, the protein is a monoclonal antibody.

In one embodiment of the present disclosure, the test device includes qualitative readout (e.g., presence/absence of a specific protein). Semi-quantitative or quantitative results are also contemplated. In some embodiments, semi-quantitative or quantitative results is achieved by including a series of test lines of mimetopes of increasing concentration. In some embodiments, the test device of the present disclosure detects active protein at least about 30, 40, 50, 60, 70, 80, 90, or 95% activity. In certain embodiments, evaluation of the signal intensity is also performed. In some embodiments, the signal is digitized and evaluated using a flatbed scanner or a CCD camera and appropriate software. In some embodiments, sensitivity is increased by use of enhancement agents such as, for example, a silver enhancer. More sensitive chemiluminescent or fluorescent labels are optionally used to increase the sensitivity as well. Free mimetope peptide, to compete for binding with the protein, is optionally included in the conjugate pad to threshold the assay, when decreased sensitivity is desired. In some embodiments, sensitivity is also controlled by adjusting, for example, the bed volume of the membrane, dimensions of the test membrane, porosity of the test membrane, position and width of the test line and control line.

In one embodiment, the disclosed test device and methods are related to determining the integrity of a protein. Proteins are increasingly used as therapeutics to treat human diseases. With their high cost, protein therapeutics are also extremely attractive targets for counterfeiting or illegal distribution. Furthermore, being large molecules, these proteins are temperature and light sensitive, and require proper handling and storage. With a complex pharmaceutical distribution chain making adequate oversight difficult, and increasing the likelihood that such problems will persist, it is imperative that a simple, inexpensive assay be developed to determine the integrity of a protein therapeutic prior to use.

In one embodiment disclosed herein, the test device allows the rapid determination of the presence of a correct protein, presence of incorrect or inactive protein, contaminated protein, no protein, and/or quantity of protein present in a sample in a single assay format.

In another embodiment disclosed herein, are device and methods for improved assessment of the therapeutic protein integrity that provides an additional level of safety and reassurance for patients fighting serious life-threatening diseases while at the same time creating a barrier to the illicit profitability of counterfeit therapeutic proteins.

In one embodiment, the disclosed test device determines the integrity of a therapeutic protein through a lateral flow assay. Lateral flow assay is an immunoassay that is used to detect various chemical or biological agents. In a typical lateral flow assay, capillary action draws fluid sample towards a zone where specific immobilized reagents reside. If the target molecule is present in the sample, the target binds to the immobilized reagent and is visualized with a detectable marker. Control lines function to confirm that the test is functional or valid, independently of whether the sample binds to the immobilized reagent.

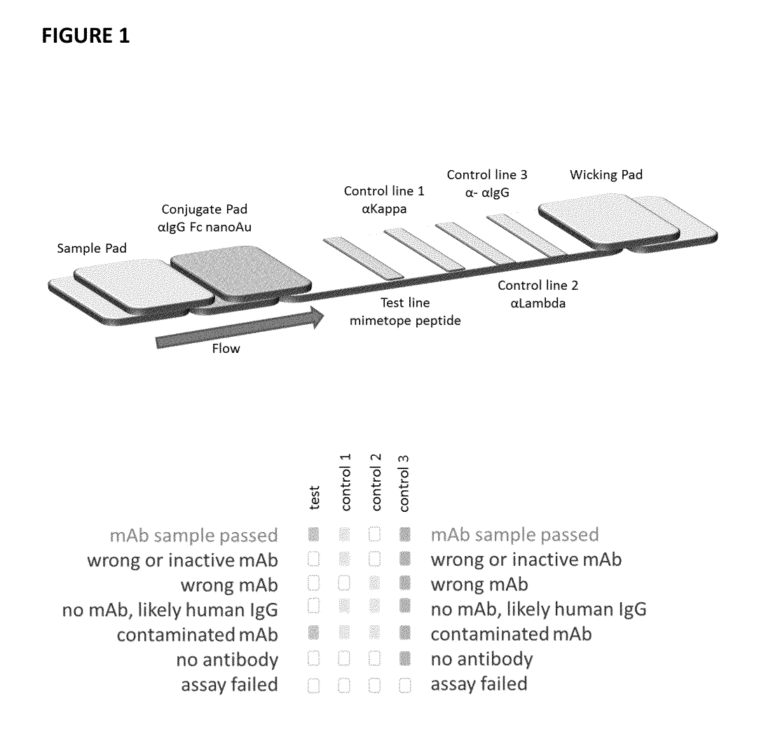

In one aspect, the test device for determining the presence or integrity of a therapeutic protein comprises a sample pad for receiving a sample comprising the therapeutic protein, a conjugate pad, and a test membrane comprising at least one test line comprising an immobilized mimetope. In some embodiments, the sample pad, conjugate pad, and test membrane are in fluid flow contact with one another. The sample pad absorbs the sample into the membrane. This is illustrated in FIG. 1.

In one embodiment, the test device is a lateral flow immunoassay. In one embodiment, the lateral flow immunoassay format is chosen from antigen sandwich assay, antibody assay, or competitive hapten assay.

In one embodiment, the sample pad of the test device further comprises a buffer for pH stabilization, a pH calibrator, a surfactant to guarantee a uniform wetting, a stabilizing polymer, and a blocker. The position of the sample pad often varies.

In one embodiment, the protein received by the sample pad is a protein therapeutic molecule. In another embodiment, the protein received by the sample pad is selected from a group consisting of: pharmaceutically or commercially relevant enzymes, receptors, receptor fusions, soluble receptors, soluble receptor fusions, antibodies (e.g., monoclonal and/or polyclonal antibodies), antigen-binding fragments of an antibody, Fc fusion proteins, SMIPs5 cytokines, hormones, regulatory factors, growth factors, coagulation/clotting factors, and antigen-binding agents. In another embodiment, the protein received by the sample pad is selected from a group consisting of Etanercept, Pegfilgrastim, Interferon β1a, and Erythropoietin.

In one embodiment, the protein is introduced to the sample pad using a dipstick format and contacting one end of the test device with the protein. In another embodiment, the protein is introduced onto the sample pad using an applicator such as, for example, a pipette, a syringe, a dropper, a spray, and others known in the art. The amount of protein received on the sample pad is preferably between about 1 and 200 μL, more preferably between about 3 and 100 μL, and most preferably between about 5 and 50 μL.

In one embodiment, the sample pad of the test device receives a protein in a fluid selected from the group consisting of buffer, saline solution, pharmaceutical composition, and biological fluid. In another embodiment, the biological fluid received by the sample pad is selected from a group consisting of: blood, urine, lacrimal fluid, sweat, saliva, and amniotic fluid. In another embodiment, the biological fluid received by the sample pad is blood.

In one embodiment, the conjugate pad of the test device comprises a detectable marker. In another embodiment, the detectable marker in the conjugate pad is capable of binding the protein that the sample pad receives. In one embodiment, the conjugate pad acts to ensure uniform transfer of the detectable marker and the protein onto the test membrane. In one embodiment, the detectable marker comprises, but not limited to, particles, luminescent labels, calorimetric labels, fluorescent labels, chemical labels, enzymes, radioactive labels, metal colloids, and chemiluminescent labels. In one embodiment, gold colloidal spheres are used. In another embodiment, other metal sols and latex microparticles are used as well. In other embodiments, photostable, color tunable nanoparticles such as carbon, selenium, or quantum dots are used as detectable markers. These detectable markers provide colorimetric indicators for reporting whether the target molecule is present. The size of the detectable markers are related to the porosity of the membrane. The markers are preferably sufficiently small to be transported along the membrane by the capillary action of the fluid. In one embodiment, the amount of detectable marker present varies depending on the size and composition of the detectable marker, the composition of the membrane, and the level of sensitivity of the assay. The detectable marker will bind to the protein to form a protein-detectable marker complex. In one embodiment, the detectable marker comprises gold colloidal spheres. In another embodiment, the detectable marker comprises a secondary protein. In some embodiments, the secondary protein is an antibody. In some embodiments, the secondary protein is conjugated to gold.

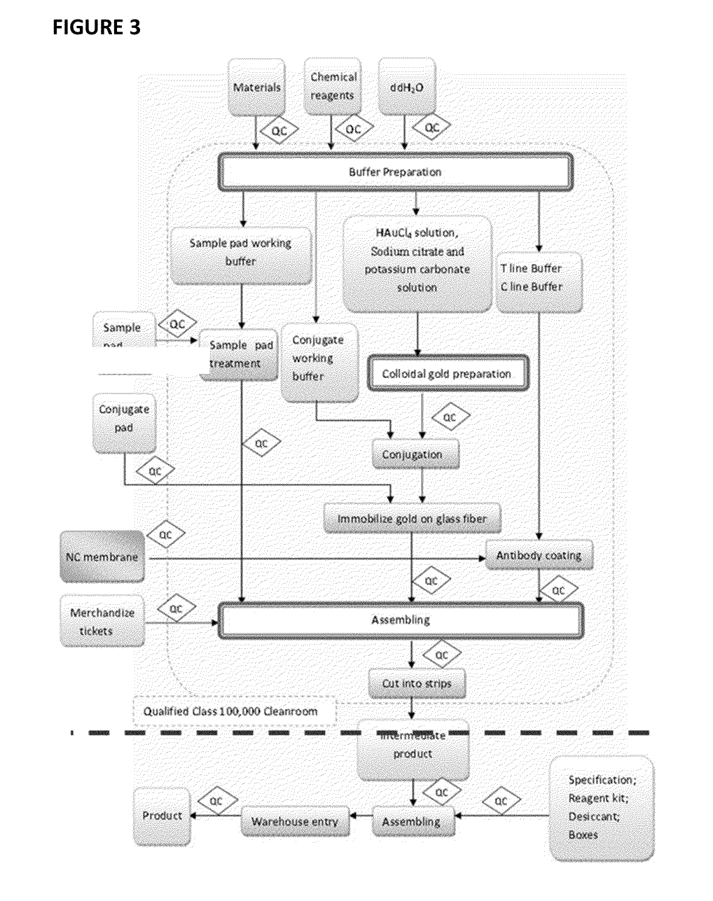

In one aspect, the present disclosure relates to methods of determining the presence or integrity of a protein comprising (a) contacting an protein with a test device, wherein the test device comprises: (i) a sample pad for receiving the protein, (ii) a conjugate pad, (iii) a test membrane comprising at least one test line comprising an immobilized mimetope, (b) determining whether at least one test line undergoes a color change, and (c) determining, based on the color change, the activity of the protein. An illustration of such a method is shown in FIG. 3.

In one embodiment, the method of determining the presence or integrity of a protein comprises a test device wherein the test device is a lateral flow assay. In another embodiment the sample pad in the method further comprises a buffer, pH calibrator, peptide, or antibody.

In one embodiment of the method disclosed herein, the sample pad of the test device receives a protein in a fluid selected from the group consisting of buffer, saline solution, pharmaceutical composition, and biological fluid. In another embodiment of the method disclosed herein, the biological fluid is selected from a group consisting of: blood, urine, lacrimal fluid, sweat, saliva, and amniotic fluid. In a preferred embodiment of the method disclosed herein, the biological fluid is blood.

In one embodiment of the method disclosed herein, the conjugate pad comprises a detectable marker. In another embodiment of the method disclosed herein, the detectable marker is selected from a group consisting of: particles, luminescent labels, calorimetric labels, fluorescent labels, chemical labels, enzymes, radioactive labels, metal colloids, and chemiluminescent labels. In another embodiment of the method disclosed herein, the detectable marker comprises gold colloidal spheres.

In one embodiment of the method disclosed herein, the detectable marker comprises a secondary protein. In one embodiment, the secondary protein is an antibody. In another embodiment of the method disclosed herein, the secondary protein is conjugated to gold.

In one embodiment of the method disclosed herein, the test membrane comprises at least one test line and at least one control line. In another embodiment of the method disclosed herein, the test membrane comprises two or more test lines. In another embodiment of the method disclosed herein, at least one test line comprises an immobilized mimetope. In another embodiment of the method disclosed herein, each test line contains an immobilized mimetope different from another test line. In another embodiment of the method disclosed herein, the immobilized mimetope mimics the native antigen epitope recognized by an antibody. In another embodiment of the method disclosed herein, the immobilized mimetope mimics the native antigen epitope recognized by an antibody.

In one embodiment of the method disclosed herein, at least one test line is upstream of at least one control line. In another embodiment of the method disclosed herein, at least one test line is downstream of at least one control line.

In one embodiment of the method disclosed herein, at least one control line comprises a light chain antibody. In another embodiment of the method disclosed herein, the light chain antibody is an anti-kappa light chain antibody. In another embodiment of the method disclosed herein, the light chain antibody is an anti-lambda light chain antibody. In another embodiment of the method disclosed herein, at least one control line comprises an antibody that binds directly to the secondary antibody of the conjugate pad.

One embodiment of the method disclosed herein, further comprises a wicking pad.

One embodiment of the present disclosure comprises a kit comprising (a) a test device for determining the presence or integrity of a protein, as described above, and (b) instructions for use thereof. In another embodiment, the kit further comprising a protein sample. In another embodiment, the kit further comprises a protein applicator. In another embodiment, the protein applicator is, for example, a pipette, a syringe, a dropper, or others known in the art.

The disclosure provided herein produces a synergistic effect and results in better recognition and binding between the protein and the mimetope. For exemplary purposes only, when a Gly-Gly-Gly-Ser linker, reactive chemistry, or biotin is added to the C-terminal serine of the mimetope, the peptide is displayed in such a way that is available for antibody binding. Attaching the mimetope to the membrane that way allows for accurate determination of the presence and purity of a sample protein or protein therapeutic. This synergism in binding was unexpected based on prior knowledge in the art. The results of this synergism are being employed in the present disclosure to determine the integrity of an antibody. These unexpected and surprising results are of significant, practical advantage to patients because administering the wrong or inactive protein results in otherwise avoidable disease progression, permanent injury, or even death.

In an exemplary aspect, the present disclosure relates to detection of proteins. Proteins are typically used in therapeutic applications, such as in the treatment of cancer.

In one embodiment, if trace amounts of a protein gives a positive result, free mimetope peptides are added to the conjugate pad. Those free mimetopes compete for the protein in the sample and its concentration will determine the threshold amount required for detection. The precise threshold will ultimately depend on the point of use, in particular whether the test is to be performed on the antibody as packaged in the shipping vials or after mixing for infusion.

In another exemplary aspect, the present disclosure relates to detection of antibodies. Antibodies are typically used in therapeutic applications, such as in the treatment of cancer. In some embodiments, as used herein, the antibody is from any origin, such as mouse, rabbit, pig, horse, dog, or human, including a chimeric antibody thereof.

In some embodiments, the antibody is a monoclonal antibody. In some embodiments, the monoclonal antibody is a bispecific monoclonal antibody. In other embodiments, the antibody is biosimilar or second generation versions of monoclonal antibodies or similar antibodies.

In some embodiments of the present disclosure, the monoclonal antibody is a bispecific monoclonal antibody such as, for example, catumaxomab, ertumaxomab, FBTA05 and TRBS07. The term “bispecific monoclonal antibody” as used herein refers to an antibody that has binding sites for two different antigens within a single antibody molecule. It will be appreciated by those skilled in the art that other molecules in addition to the canonical antibody structure may be constructed with two binding specificities.

In one embodiment of the present disclosure, the test device includes qualitative readout (e.g., presence/absence of a specific antibody). Semi-quantitative or quantitative results are also contemplated. In some embodiments, semi-quantitative or quantitative results is achieved by including a series of test lines of mimetopes of increasing concentration. In some embodiments, the test device of the present disclosure detects active antibody at least about 30, 40, 50, 60, 70, 80, 90, or 95% activity. In certain embodiments, evaluation of the signal intensity is also performed. In some embodiments, the signal is digitized and evaluated using a flatbed scanner or a CCD camera and appropriate software. In some embodiments, sensitivity is increased by use of enhancement agents such as, for example, a silver enhancer. More sensitive chemiluminescent or fluorescent labels are optionally used to increase the sensitivity as well. Free mimetope peptide, to compete for binding with the antibody, is optionally included in the conjugate pad to threshold the assay, when decreased sensitivity is desired. In some embodiments, sensitivity is also controlled by adjusting, for example, the bed volume of the membrane, dimensions of the test membrane, porosity of the test membrane, position and width of the test line and control line.

In one embodiment, the disclosed test device and methods are related to determining the integrity of an antibody. Antibodies are increasingly used as therapeutics to treat human diseases such as cancer and autoimmunity. With their high cost, antibodies are also extremely attractive targets for counterfeiting or illegal distribution. Furthermore, being large proteins, antibodies are temperature and light sensitive, and require proper handling and storage. With a complex pharmaceutical distribution chain making adequate oversight difficult, and increasing the likelihood that such problems will persist, it is imperative that a simple, inexpensive assay be developed to determine the integrity of an antibody prior to use.

In one embodiment disclosed herein, the test device allows the rapid determination of the presence of a correct antibody, presence of incorrect or inactive antibody, presence of non-antibody protein, contaminated antibody, no antibody, and/or quantity of antibody present in a sample in a single assay format.

In another embodiment disclosed herein, are device and methods for improved assessment of the antibody integrity that provides an additional level of safety and reassurance for patients fighting serious life-threatening diseases while at the same time creating a barrier to the illicit profitability of counterfeit antibodies.

In one embodiment, the disclosed test device determines the integrity of an antibody through a lateral flow assay. Lateral flow assay is an immunoassay that is used to detect various chemical or biological agents. In a typical lateral flow assay, capillary action draws fluid sample towards a zone where specific immobilized reagents reside. If the target molecule is present in the sample, the target binds to the immobilized reagent and is visualized with a detectable marker. Control lines function to confirm that the test is functional or valid, independently of whether the sample binds to the immobilized reagent.

In one aspect, the test device for determining the presence or integrity of an antibody comprises a sample pad for receiving a sample comprising the antibody, a conjugate pad, and a test membrane comprising at least one test line comprising an immobilized mimetope. In some embodiments, the sample pad, conjugate pad, and test membrane are in fluid flow contact with one another. The sample pad absorbs the sample into the membrane. This is illustrated in FIG. 1.

In one embodiment, the test device is a lateral flow immunoassay. In one embodiment, the lateral flow immunoassay format is chosen from antigen sandwich assay, antibody assay, or competitive hapten assay.

In one embodiment, the sample pad of the test device further comprises a buffer for pH stabilization, a pH calibrator, a surfactant to guarantee a uniform wetting, a stabilizing polymer, and a blocker. The position of the sample pad often varies.

In one embodiment, the antibody received by the sample pad is a monoclonal antibody. In another embodiment, the monoclonal antibody received by the sample pad is selected from a group consisting of: bevacizumab, trastuzumab, rituximab, abciximab, adalimumab, alemtuzumab, basiliximab, belimumab, brentuximab, vedotin, canakinumab, cetuximab, certolizumab pegol, daclizumab, denosumab, eculizumab, efalizumab, gemtuzumab, golimumab, ibritumomab, tiuxetan, infliximab, ipilimumab, muromonab-CD3, natalizumab, ofatumumab, omalizumab, palivizumab, panitumumab, ranibizumab, raxibacumab, tocilizumab, tositumomab, and ustekinumab. In another embodiment, the monoclonal antibody received by the sample pad is a bispecific monoclonal antibody. In another embodiment, the bispecific monoclonal antibody received by the sample pad is selected from a group consisting of: catumaxomab, ertumaxomab, FBTA05, and TRBS07.

In one embodiment, the antibody is introduced to the sample pad using a dipstick format and contacting one end of the test device with the antibody. In another embodiment, the antibody is introduced onto the sample pad using an applicator such as, for example, a pipette, a syringe, a dropper, a spray, and others known in the art. The amount of antibody received on the sample pad is preferably between about 1 and 200 μL, more preferably between about 3 and 100 μL, and most preferably between about 5 and 50 μL.

In one embodiment, the sample pad of the test device receives an antibody in a biological fluid. In another embodiment, the biological fluid received by the sample pad is selected from a group consisting of: blood, urine, lacrimal fluid, sweat, saliva, and amniotic fluid. In another embodiment, the biological fluid received by the sample pad is blood.

In one embodiment, the conjugate pad of the test device comprises a detectable marker. In another embodiment, the detectable marker in the conjugate pad is capable of binding the antibody that the sample pad receives. In one embodiment, the conjugate pad acts to ensure uniform transfer of the detectable marker and the antibody onto the test membrane. In one embodiment, the detectable marker comprises, but not limited to, particles, luminescent labels, calorimetric labels, fluorescent labels, chemical labels, enzymes, radioactive labels, metal colloids, and chemiluminescent labels. In one embodiment, gold colloidal spheres are used. In another embodiment, other metal sols and latex microparticles are used as well. In other embodiments, photostable, color tunable nanoparticles such as carbon, selenium, or quantum dots are used as detectable markers. These detectable markers provide colorimetric indicators for reporting whether the target molecule is present. The size of the detectable markers are related to the porosity of the membrane. The markers are preferably sufficiently small to be transported along the membrane by the capillary action of the fluid. In one embodiment, the amount of detectable marker present varies depending on the size and composition of the detectable marker, the composition of the membrane, and the level of sensitivity of the assay. The detectable marker will bind to the antibody to form an antibody-detectable marker complex. In one embodiment, the detectable marker comprises gold colloidal spheres. In another embodiment, the detectable marker comprises a secondary antibody. In some embodiments, the secondary antibody is conjugated to gold.

In one embodiment, the test membrane in the test device comprises at least one test line and at least one control line. In another embodiment, the test membrane comprises two or more test lines. In another embodiment, at least one test line comprises an immobilized mimetope. In another embodiment, each test line contains an immobilized mimetope different from another test line. In some embodiments, the immobilized mimetope mimics the native antigen epitope recognized by an antibody.

In one embodiment, at least one test line is upstream of at least one control line. In another embodiment, at least one test line is downstream of at least one control line.

In one embodiment, at least one control line comprises a light chain antibody. In some embodiments, the light chain antibody is an anti-kappa light chain antibody. In another embodiment, the light chain antibody is an anti-lambda light chain antibody. In other embodiments, the light chain antibody is a light chain antibody known in the art.

In one embodiment, the at least one control line of the disclosed test membrane comprises an antibody that binds directly to the secondary antibody of the conjugate pad such as, for example, anti-IgG. In some of these embodiments, the anti-IgG acts as a non-specific control and turns positive regardless of the presence or absence of antibody within the test sample. In some embodiments, the test device consists of only one control line. In one embodiment, the test device of the present disclosure consists of two control lines. In another embodiment, the test device of the present disclosure consists of three control lines. It is also contemplated that the test device consists of more than three control lines.

In some embodiments, the test device further comprises a wicking pad.

In another aspect, the present disclosure relates to methods of determining the presence or integrity of an antibody comprising (a) contacting an antibody with a test device, wherein the test device comprises: (i) a sample pad for receiving the antibody, (ii) a conjugate pad, (iii) a test membrane comprising at least one test line comprising an immobilized mimetope, (b) determining whether at least one test line undergoes a color change, and (c) determining, based on the color change, the activity of the antibody. An illustration of such a method is shown in FIG. 3.

In one embodiment, the method of determining the presence or integrity of an antibody comprises a test device wherein the test device is a lateral flow immunoassay. In another embodiment the sample pad in the method further comprises a buffer, pH calibrator, peptide, or antibody.

In another embodiment of the method disclosed herein, the antibody is a monoclonal antibody. In some embodiments, the monoclonal antibody in this method is selected from a group consisting of: bevacizumab, trastuzumab, rituximab, abciximab, adalimumab, alemtuzumab, basiliximab, belimumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, raxibacumab, tocilizumab, tositumomab, and ustekinumab.

In another embodiment of the method disclosed herein, the monoclonal antibody is a bispecific monoclonal antibody. In some embodiments of the method disclosed herein, the bispecific monoclonal antibody is selected from a group consisting of: catumaxomab, ertumaxomab, FBTA05, and TRB S07.

In one embodiment of the method disclosed herein, the sample pad receives an antibody in a biological fluid. In another embodiment of the method disclosed herein, the biological fluid is selected from a group consisting of: blood, urine, lacrimal fluid, sweat, saliva, and amniotic fluid. In a preferred embodiment of the method disclosed herein, the biological fluid is blood.

In one embodiment of the method disclosed herein, the conjugate pad comprises a detectable marker. In another embodiment of the method disclosed herein, the detectable marker is selected from a group consisting of: particles, luminescent labels, calorimetric labels, fluorescent labels, chemical labels, enzymes, radioactive labels, metal colloids, and chemiluminescent labels. In another embodiment of the method disclosed herein, the detectable marker comprises gold colloidal spheres.

In one embodiment of the method disclosed herein, the detectable marker comprises a secondary antibody. In another embodiment of the method disclosed herein, the secondary antibody is conjugated to gold.

In one embodiment of the method disclosed herein, the test membrane comprises at least one test line and at least one control line. In another embodiment of the method disclosed herein, the test membrane comprises two or more test lines. In another embodiment of the method disclosed herein, at least one test line comprises an immobilized mimetope. In another embodiment of the method disclosed herein, each test line contains an immobilized mimetope different from another test line. In another embodiment of the method disclosed herein, the immobilized mimetope mimics the native antigen epitope recognized by an antibody. In another embodiment of the method disclosed herein, the immobilized mimetope mimics the native antigen epitope recognized by an antibody.

In one embodiment of the method disclosed herein, at least one test line is upstream of at least one control line. In another embodiment of the method disclosed herein, at least one test line is downstream of at least one control line.

In one embodiment of the method disclosed herein, at least one control line comprises a light chain antibody. In another embodiment of the method disclosed herein, the light chain antibody is an anti-kappa light chain antibody. In another embodiment of the method disclosed herein, the light chain antibody is an anti-lambda light chain antibody. In another embodiment of the method disclosed herein, at least one control line comprises an antibody that binds directly to the secondary antibody of the conjugate pad.

One embodiment of the method disclosed herein, further comprises a wicking pad.

One embodiment of the present disclosure comprises a kit comprising (a) a test device for determining the presence or integrity of an antibody, as described above, and (b) instructions for use thereof. In another embodiment, the kit further comprising an antibody sample. In another embodiment, the kit further comprises an antibody applicator. In another embodiment, the antibody applicator is, for example, a pipette, a syringe, a dropper, or others known in the art.

In some embodiments, the test device of the present disclosure has a protective cover. In some embodiments, the protective cover is formed of any material which is impervious to water, and is preferably translucent or transparent. In certain embodiments, the protective covering comprises a single or multiple layers. Preferable materials for use in the protective covering include optically transmissive materials such as polyamide, polyester, polyethelene, acrylic, glass, or similar materials. The protective covering is optionally clear (i.e., see-through) or not clear (i.e., opaque) depending on the method of detection used. In a preferable embodiment, a protective covering is optically clear polyester.

In some embodiments, the present disclosure include mimetope peptides comprising about 5 to 15 amino acids or about 7 to 12 amino acids, that bind to the antigen-binding site of the antibody and are competed by the natural ligand.

In one embodiment, the mimetope peptides disclosed herein are generated as described in WO 2009/121024, the disclosure of which is incorporated herein by reference. The mimetope peptides are selected using phage displayed peptide libraries. These mimetopes are then modified for use in, for example, lateral flow assays. In one embodiment, mimetopes contain additional amino acids to alter or modify mimetope characteristics. In some embodiments, mimetopes contain cysteines flanking the mimetope sequence to increase stability of the mimetope through disulfide bond formation. In one embodiment, mimetopes are attached to a solid substance such as, for example, the test membrane. In one embodiment, mimetopes are attached to the test membrane directly or synthesized directly on the test membrane. In other embodiments, linkers such as, for example, Gly-Gly-Gly-Ser linkers, are added to mimetopes to ensure the mimetope are displayed on the solid substance in such a way to be available for antibody binding. In one embodiment, the linker is about 1, 2, 3, 4, 5, 6, 7, 8, 9, or 10 amino acids in length. In other embodiments, reactive chemistry or biotin is added to the C-terminal serine. If amine reactive chemistries are used to attach the mimetopes to the solid substance and there are no amine containing side chains within the peptide sequence, the N-terminus is acetylated and a C-terminal lysine added to provide the reactive amine. Additionally, linear and disulfide constrained peptide libraries are also used. In one embodiment, mimetopes are expressed as a fusion with a carrier protein such as KLH.

The disclosure provided herein produces a synergistic effect and results in better recognition and binding between the antibody and the mimetope. For exemplary purposes only, when a Gly-Gly-Gly-Ser linker, reactive chemistry, or biotin is added to the C-terminal serine of the mimetope, the peptide is displayed in such a way that is available for antibody binding. Attaching the mimetope to the membrane that way allows for accurate determination of the presence and purity of a sample antibody. This synergism in binding was unexpected based on prior knowledge in the art. The results of this synergism are being employed in the present disclosure to determine the integrity of an antibody. These unexpected and surprising results are of significant, practical advantage to patients because administering the wrong or inactive antibody results in otherwise avoidable disease progression, permanent injury, or even death.

In an exemplary aspect, the present disclosure relates to detection of monoclonal antibodies. Monoclonal antibodies are typically used in therapeutic applications, such as in the treatment of cancer. For exemplary purposes only, Table 1 lists some common monoclonal antibodies and the corresponding mimetope peptides.

TABLE 1
Mimetope peptide sequence for some antibody

| Target antibody | Mimetope sequence |
|---|---|
| Rituximab | ACPYSNPSLC |
| Alemtuzumab | ACGSLSPSSC |
| Bevacizumab | ACFLRSGLPC |
| Trastuzumab | ACVDHHLDHC |

In one embodiment, if trace amounts of antibody gives a positive result, free mimetope peptides are added to the conjugate pad. That free mimetope competes for the antibody in the sample and its concentration will determine the threshold amount required for detection. The precise threshold will ultimately depend on the point of use, in particular whether the test is to be performed on the antibody as packaged in the shipping vials or after mixing for infusion.

Embodiments of the disclosure are further described with reference to the accompanying drawings.

The following examples are provided to further illustrate the embodiments of the present disclosure, but are not intended to limit the scope of the disclosure. While they are typical of those that might be used, other procedures, methodologies or techniques known to those skilled in the art may alternatively be used.

### Example 1: Identification and Testing of Mimetopes

While the following assay reagent protocol is described using specifically identified reagents, such as specific phage-displayed libraries and generation of specifically identified peptides, the methods described herein is utilized to generate peptides including mimetopes that interact with any antibody.

Peptide mimetopes were identified and generated as described in the disclosure of WO2009/121024 and in Sanchez et al., Cancer Chemther. Pharmacol. (2010) 66:919-925, which is incorporated herein by reference in its entirety. Phage-display libraries of peptide mimetopes were screened against the monoclonal antibodies, including, but not limited to, bevacizumab, tratuzumab, and rituximab. Short peptides of 7 to 12 amino acids were screened and were selected from a library that contain cysteines flanking the peptide mimetope sequence. To ensure that the peptide was displayed on the test membrane in such a way as to be available for antibody binding, a glycine-glycine-glycine-serine linker was added and the reactive chemistry or biotin added to the C-terminal serine. If amine reactive chemistries were used for conjugation and there were no amine containing side chains within the peptide sequence, the N-terminus was acetylated and a C-terminal lysine added to provide the reactive amine.

To confirm that a synthetic peptide captures the antibody, ELISAs were performed. Peptides biotinylated at the C-terminus were coated onto streptavidin coated plates and then incubated with a dilution serious of antibody. Binding was detected via a peroxidase labeled secondary anti-Fc antibody. Specificity was confirmed by lack of signal from either total Ig or using a different antibody. Native antigen competition assays were performed to confirm that the validated mimetope peptide was binding to the antibody binding site and thus detecting only active antibody. For antigens that were cell membrane bound, competition was shown by flow cytometry. The antibody was fluorescently labeled and the cell staining evaluated with or without a molar excess of the presumed mimetope as well as control peptides.

Validated peptides detects >100 ng/ml of the antibody. For example, the sensitivity was 4 ug/ml for rituximab. Specificity was confirmed by ensuring that there was no signal from either total human Ig or a different antibody. Competition for antibody binding between the peptide and the native antigen confirmed that the mimetope peptide was binding the antibody-antigen binding site and thus detecting only active antibody. As another example, bevacizumab, whose target antigen is the soluble protein VEGF, competitive ELISAs were used in which the peptide of VEGF was coated to the plate, and the other was used in solution to compete for antibody binding.

### Example 2: Validation of Rituximab Binding Mimetope Peptide in the Lateral Flow Assay

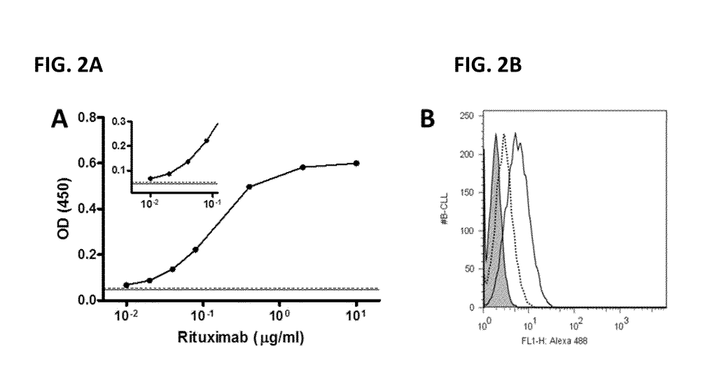

FIG. 2 illustrates one embodiment of the lateral flow assay validating that rituximab binding mimetope peptide function in immunoassays and competes with the native antigen (CD20). (A) is a standard curve for rituximab by peptide based ELISA. Biotinylayed peptides were bound onto neutravidin coated ELISA plates. Rituximab was diluted in TBST. Each value shows the mean (±S.D.) of triplicates. The solid line indicates the mean of the buffer control and the dashed line represents the mean +10 times the SD of the buffer control. (B) Peptide inhibition of CLL cell staining. Fluorescently labeled rituximab was incubated with primary CLL cells and evaluated by flow cytometry (solid line). Weak staining for CD20 was observed since CLL cells are known to have low levels of CD20. When peptide RTX-10 was added at a large molar excess (dashed lines), the cell labeling is largely abrogated. Control peptides had no effect (not shown). The shaded histogram represents CLL cells incubated with fluorescently labeled normal human IgG.

### Example 3: Validation of Etanercept and Erythropoetin Binding Mimetope Peptide in the Lateral Flow Assay

Peptide mimetopes are identified and generated as described in Example 1. Phage-display libraries of peptide mimetopes are screened against protein molecules, including, but not limited to, protein therapeutics such as Etanercept and Erythropoetin. The identified peptide mimetope is displayed on the test membrane in such a way as to be available for protein binding, such as by adding a glycine-glycine-glycine-serine linker, and/or adding the reactive chemistry or biotin to the C-terminal serine. If amine reactive chemistries are used for conjugation and there are no amine containing side chains within the peptide sequence, the N-terminus is acetylated and a C-terminal lysine is added to provide the reactive amine.

To confirm that a synthetic peptide captures the protein Etanercept and/or Erythropoetin, sandwich type assays, lateral flow or otherwise, are performed.

### Example 4: Validation of Test Device

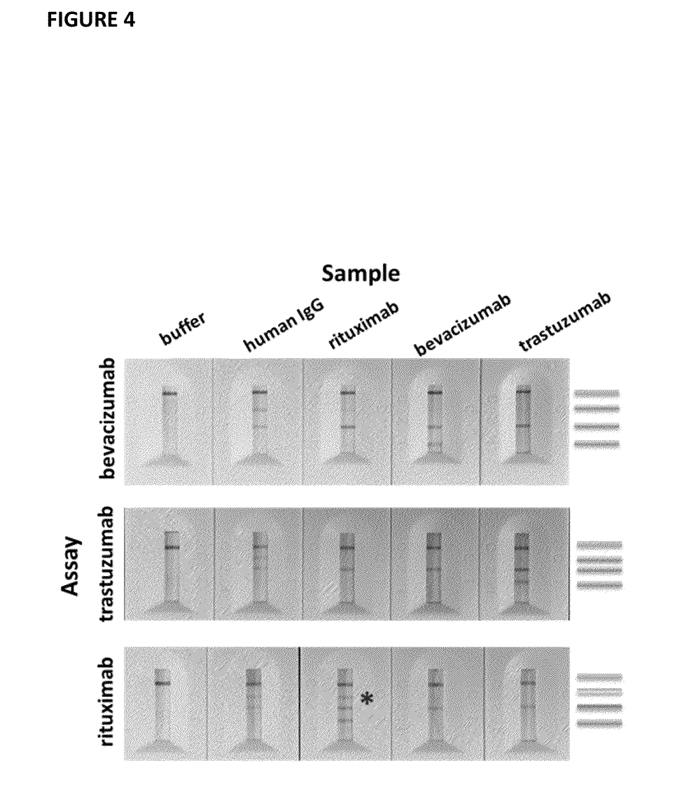

FIG. 4 shows the results of an independent double-blinded test was performed on the test device. Samples of antibodies, including, but not limited to, bevacizumab, tratuzumab, rituximab, and total human IgG (IVIG), were prepared at 0.1 mg/ml and control saline sample. 240 numbered LFA tests and samples were provided such that each mAb assay was tested with 20 samples of each mAb and 10 samples of human IgG and buffer. This was a double-blinded test—the laboratory technicians who ran the assays knew neither the sample's identity nor the type of LFA, both of which were randomly ordered, but recorded the results they observed. The test lines were accurate when the tests ran properly. Only two bevacizumab assays failed to run. There was one false positive on the kappa line of a rituximab LFA but several false positive lambda lines, suggesting that the anti-lambda antibody used was not optimal. No false negatives other than the two defective assays were observed.

### Example 5: Range of the Lateral Flow Assay

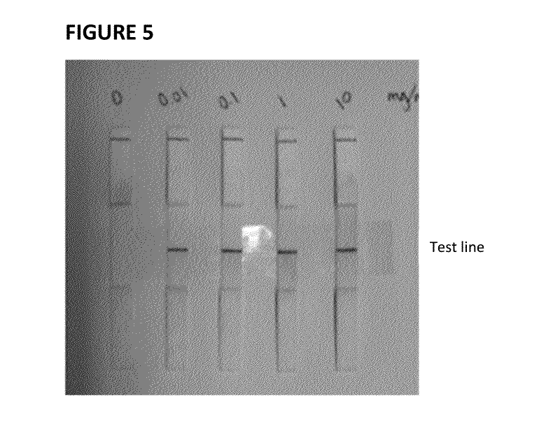

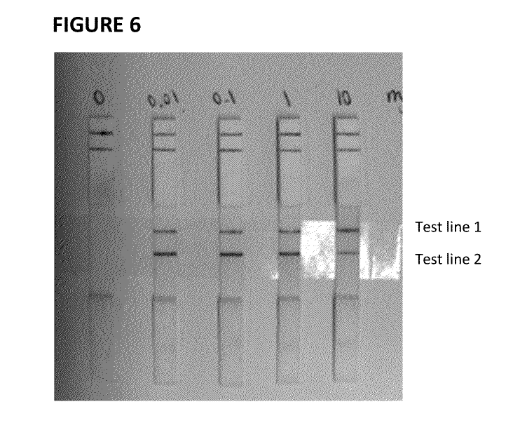

FIG. 5 and FIG. 6 illustrate two embodiments that the lateral flow assay disclosed herein has wide dynamic range. FIG. 5 shows that samples tested were Rituximab at 0.0 mg/ml (negative control), 0.01 mg/ml, 0.1 mg/ml, 1.0 mg/ml, and 10 mg/ml (vial concentration). With increasing Rituximab concentration, the test line gets more intense. FIG. 6 illustrate one embodiment of the sandwich lateral flow assay with two test lines. The test lines comprise peptide mimetope striped at two different concentrations on the membrane. Samples tested were Rituximab at 0.0 mg/ml (negative control), 0.01 mg/ml, 0.1 mg/ml, 1.0 mg/ml, and 10 mg/ml (vial concentration). With increasing Rituximab concentration, the lower test line gets less intense whereas the upper test line gets more intense.

While the present disclosure has been described and illustrated herein, it is intended that the specification and examples be considered as exemplary only, with the true scope and spirit of the invention being indicated by the following claims.

What is claimed is:

1. A test device for determining the presence or integrity of a biologic comprising
a. a sample pad for receiving the biologic,
b. a conjugate pad, and
c. a test membrane comprising at least one test line comprising an immobilized mimetope.

2. The test device of claim 1, wherein the test device is a lateral flow immunoassay.

3. The test device of claim 1, wherein the sample pad further comprises a buffer, pH calibrator, peptide, or antibody.

4. The test device of claim 1, wherein the biologic is a protein.

5. The test device of claim 4, wherein the protein is an antibody.

6. The test device of claim 5, wherein the antibody is a monoclonal antibody.

7. The test device of claim 6, wherein the monoclonal antibody is selected from a group consisting of: bevacizumab, trastuzumab, rituximab, abciximab, adalimumab, alemtuzumab, basiliximab, belimumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, raxibacumab, tocilizumab, tositumomab, and ustekinumab.

8. The test device of claim 6, wherein the monoclonal antibody is a bispecific monoclonal antibody.

9. The test device of claim 8, wherein the bispecific monoclonal antibody is selected from a group consisting of: catumaxomab, ertumaxomab, FBTA05, and TRBS07.

10. The test device of claim 1, wherein the sample pad receives an antibody in a fluid selected from the group consisting of buffer, saline solution, pharmaceutical composition, and biological fluid.

11. The test device of claim 10, wherein the biological fluid is selected from a group consisting of: blood, urine, saliva, lacrimal fluid, sweat, and amniotic fluid.

12. The test device of claim 11, wherein the biological fluid is blood.

13. The test device of claim 1, wherein the conjugate pad comprises a detectable marker.

14. The test device of claim 13, wherein the detectable marker is selected from a group consisting of: particles, luminescent labels, calorimetric labels, fluorescent labels, chemical labels, enzymes, radioactive labels, metal colloids, and chemiluminescent labels.

15. The test device of claim 13, wherein the detectable marker comprises gold colloidal spheres.

16. The test device of claim 13, wherein the detectable marker comprises a secondary antibody.

17. The test device of claim 16, wherein the secondary antibody is conjugated to gold.

18. The test device of claim 1, wherein the test membrane comprises at least one test line and at least one control line.

19. The test device of claim 18, wherein the test membrane comprises two or more test lines.

20. The test device of claim 18, wherein at least one test line comprises an immobilized mimetope.

21. The test device of claim 20, wherein each test line contains an immobilized mimetope different from another test line.

22. The test device of claim 21, wherein the immobilized mimetope mimics the native antigen epitope recognized by an antibody.

23. The test device of claim 18, wherein the at least one test line is upstream of the at least one control line.

24. The test device of claim 18, wherein the at least one test line is downstream of the at least one control line.

25. The test device of claim 18, wherein at least one control line comprises a light chain antibody.

26. The test device of claim 25, wherein the light chain antibody is an anti-kappa light chain antibody.

27. The test device of claim 25, wherein the light chain antibody is an anti-lambda light chain antibody.

28. The test device of claim 18, wherein the at least one control line comprises an antibody that binds directly to the secondary antibody of the conjugate pad.

29. The test device of claim 1, further comprising a wicking pad.

30. A method of determining the integrity of a biologic comprising
(a) contacting the biologic with a test device, wherein the test device comprises: (i) a sample pad for receiving the biologic, (ii) a conjugate pad, (iii) a test membrane comprising at least one test line comprising an immobilized mimetope,
(b) determining whether at least one test line undergoes a color change,
(c) determining, based on the color change, the activity of the biologic.

31. The method of claim 30, wherein the test device is a lateral flow immunoassay.

32. The method of claim 30, wherein the sample pad further comprises a buffer, pH calibrator, peptide, or antibody.

33. The test device of claim 30, wherein the biologic is a protein.

34. The test device of claim 33, wherein the protein is an antibody.

35. The method of claim 34, wherein the antibody is a monoclonal antibody.

36. The method of claim 35 wherein the monoclonal antibody is selected from a group consisting of: bevacizumab, trastuzumab, rituximab, abciximab, adalimumab, alemtuzumab, basiliximab, belimumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, raxibacumab, tocilizumab, tositumomab, and ustekinumab.

37. The method of claim 35, wherein the monoclonal antibody is a bispecific monoclonal antibody.

38. The method of claim 37, wherein the bispecific monoclonal antibody is selected from a group consisting of: catumaxomab, ertumaxomab, FBTA05, and TRBS07.

39. The method of claim 30, wherein the sample pad receives an antibody in a fluid selected from the group consisting of buffer, saline solution, pharmaceutical composition, and biological fluid.

40. The method of claim 39, wherein the biological fluid is selected from a group consisting of: blood, urine, saliva, lacrimal fluid, sweat, and amniotic fluid.

41. The method of claim 40, wherein the biological fluid is blood.

42. The method of claim 30, wherein the conjugate pad comprises a detectable marker.

43. The method of claim 42, wherein the detectable marker is selected from a group consisting of: particles, luminescent labels, calorimetric labels, fluorescent labels, chemical labels, enzymes, radioactive labels, metal colloids, and chemiluminescent labels.

44. The method of claim 42, wherein the detectable marker comprises gold colloidal spheres.

45. The method of claim 42, wherein the detectable marker comprises a secondary antibody.

46. The method of claim 45 wherein the secondary antibody is conjugated to gold.

47. The method of claim 30, wherein the test membrane comprises at least one test line and at least one control line.

48. The method of claim 47, wherein the test membrane comprises two or more test lines.

49. The method of claim 47, wherein the at least one test line comprises an immobilized mimetope.

50. The method of claim 48, wherein each test line contains an immobilized mimetope different from another test line.

51. The method of claim 49, wherein the immobilized mimetope mimics the native antigen epitope recognized by an antibody.

52. The method of claim 47, wherein the at least one test line is upstream of the at least one control line.

53. The method of claim 47, wherein the at least one test line is downstream of the at least one control line.

54. The method of claim 47, wherein at least one control line comprises a light chain antibody.

55. The method of claim 54, wherein the light chain antibody is an anti-kappa light chain antibody.

56. The method of claim 54, wherein the light chain antibody is an anti-lambda light chain antibody.

57. The method of claim 47, wherein at least one control line comprises an antibody that binds directly to the secondary antibody of the conjugate pad.

58. The method of claim 30, further comprising a wicking pad.

59. A kit comprising: (a) a test device for determining the integrity of a biologic, the test device comprising:(i) a sample receiving pad, (ii) a conjugate pad, (iii) a test membrane, the test membrane further comprising at least one test line and at least one control line; and (b) instructions for use thereof.

60. The kit of claim 59, further comprising a biologic sample.

61. The kit of claim 59, further comprising a biologic applicator.
